(19) United States
(12) Patent Application Publication
(10) Pub. No.: US 2005/0090901 A1
(43) Pub. Date: Apr. 28, 2005

(54) Intervertebral disk prosthesis or nucleus replacement prosthesis

(76) Inventor: Armin Studer, Winterthur (CH)

Correspondence Address: RANKIN, HILL, PORTER & CLARK LLP, 4080 ERIE STREET, WILLOUGHBY, OH, 44094-7836, US

(21) Appl. No.: 10/497,712

(22) PCT Filed: Dec. 5, 2001
(86) PCT No.: PCT/CH01/00700

(51) Int. Cl. A61F 2/44

(52) U.S. Cl. 623/17.12; 623/17.16

(57) ABSTRACT

An intervertebral disk prosthesis or nucleus replacement prosthesis includes a bio-compatible pouch (1) receiving a curable, flowable material (2). The curable flowable material includes monomers, comonomers, homopolymers, oligomers or mixtures thereof. Curing the curable, flowable material introduced into the pouch (1) takes place in situ after the prosthesis is installed in the intervertebral space between two adjacent vertebras.

The present invention relates to an intervertebral disk prosthesis or nucleus replacement prosthesis defined in the preamble of claim 1.

A substantial number of such intervertebral disk prostheses is already known in the state of the art, said prostheses however all being prefabricated and requiring implantation in the prefabricated, comparatively bulky state into the intervertebral space.

The above cited state of the art is merely cited to discuss the background of the present invention, but it does not imply that said cited state of the art was in fact published or known to the public at the time of this application or its priority.

The objective of the present invention is to create an intervertebral disk prosthesis or nucleus replacement prosthesis allowing implantation in a comparatively dimensionally compacted stated into the intervertebral space and, after being filled with a curable, flowable substance, to be solidified by a curing procedure.

The present invention solves the above problem using an intervertebral disk prosthesis comprising the features of claim 1.

The still empty pouch of the intervertebral disk prosthesis is easily inserted in its collapsed state into the intervertebral space and then may be filled by means of a syringe and an appropriate cannula with a flowable mixture of monomers. The pouch (or balloon) may be fitted with a special surface and/or thickness and/or a special material such as polycarbonate urethane (PCU) or a polycarbonate so it shall make contact by its appropriate sides with the upper plates of the adjacent vertebras.

This design offers the advantages that the contact surfaces of the two upper plates (cartilage layer) of the adjacent vertebras shall entail optimal conditions of sliding, biocompatibility, rigidity etc. at the involved motions (rotation, extension, flexion).

By selecting appropriate pressurization, said pouch may be filled with the polymerizable mixture of monomers to such an extent that the intervertebral disk height shall once again be the appropriate anatomical initial height. In this procedure, the said material may be introduced into the pouch at an excess pressure of less than 3 atmospheres, preferably no more than 1.1 atmosphere.

However said material also may be introduced into the pouch in the absence of substantial excess pressure when the affected vertebras are kept spaced apart using appropriate implements.

By inserting a light guide (for instance an optical fiber cable) into the pouch, i.e. into its aperture, the polymerizable material illustratively may be photo-polymerized using blue light (for instance of 340 nm wavelength). As regards aqueous monomer solutions, polymer cross-linking may result in a hydrogel.

Such a result offers the advantage that in the event of stress on the body, the hydrogel may release water, whereas in the case of the body at rest, it may absorb water. In this manner a damping effect is attained, furthermore the possibility to restore the intervertebral disk to its initial height. In a another preferred embodiment of the present invention, the pouch is double-walled and the curable, flowable material containing monomers, comonomers, homopolymers, oligomers or mixtures thereof is introduced between said two walls, as a result of which the center of the intervertebral disk prosthesis is hollow. The freely selectable size of said cavity allows additional control of Implant flexibility.

In yet another embodiment mode of the present invention, the pouch is chemically identical with the curable, flowable material it contains, as a result of which said latter material may combine with the pouch material.

In a further embodiment mode of the invention, the pouch consists of a memory-effect substance, as a result of which it assumes the geometric shape previously stored at body temperature.

In yet another embodiment mode of the invention, the curable, flowable material contains a polymerization catalyst and preferably a polymerization accelerator.

In yet another preferred embodiment of the invention, the curable, flowable material contains a photo-initiator, preferably a radicals-generating photo-initiator, where said photoinitiator preferably absorbs light in the 340 to 420 nm range. The photo-initiator may be phosphine oxide, preferably an acylphosphine oxide. The phospine oxide may be copolymerized with dimethylacrylamide. Blue light polymerization offers the advantage over auto-polymerization that higher heat dissipation that might destroy the protein molecule will not take place. Moreover a light guide irradiating the blue light into the balloon may be handled free of danger. The frequency and duration of blue light irradiation may be set merely by controlling the light source.

The monomers, comonomers, homopolymers, oligomers or mixtures that are contained in the curable, flowable material, may be appropriately selected from the group of
(a) polyethylene glycols, preferably polyethylene glycol diacrylates;
(b) N-vinyl pyrrolidones; and
(c) vinyls, preferably vinyl alcohols; and
(d) styrenes.

The polymers prepared thereby may be varied within wide ranges as regards their elasticities.

Advantageously the curable flowable material contains 30 to 160% by wt, preferably 40 to 90% by wt water. A proportion of 45 to 55% by wt water is especially appropriate. By determining how much water the polymerized material—especially when it is a hydrogel—subsequently shall absorb—the swelling factor—, the additional traction on the spine segment also may be controlled.

A method for manufacturing the intervertebral disk prosthesis or nucleus replacement prosthesis includes the following steps:
(a) implanting a bio-compatible pouch into the intervertebral space between two adjacent vertebras,
(b) introducing a curable, flowable material containing monomers, comonomers, oligomers or mixtures thereof inside the implanted, bio-compatible pouch, the filled pouch remaining centered in the intervertebral space, and
(c) curing in situ the curable, flowable material in the pouch.

In one variation of the method of the present invention, the pouch may be inflated with air between steps (a) and (b). By means of this preliminary traction, the tractive capacity of the spine segment may be checked.

In a further variation of the method of the present invention, the pouch may be filled with an x-ray contrast means. Said contrast means makes visible the pouch in the spine segment by means of an image converter. This feature allows a check on the proper pouch position.

The said material may be cured by auto-polymerization or by photo-polymerization, preferably using visible or ultraviolet light.

The invention and further implementations of it are elucidated below by means of several illustrative embodiment modes which are shown in partly schematic manner.

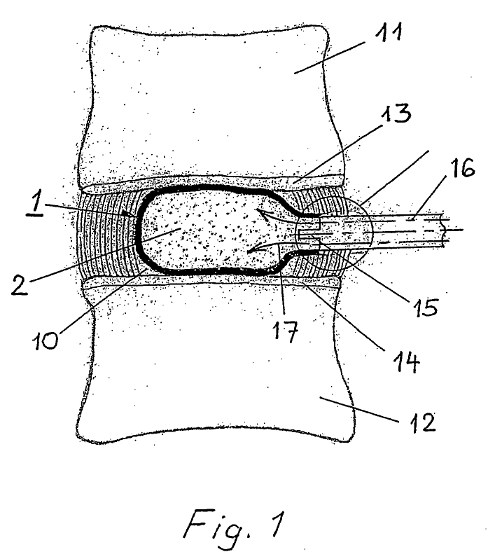

FIG. 1 is a longitudinal section of an intervertebral disk prosthesis implanted between two adjacent vertebras while the pouch is being filled with a curable and flowable material;

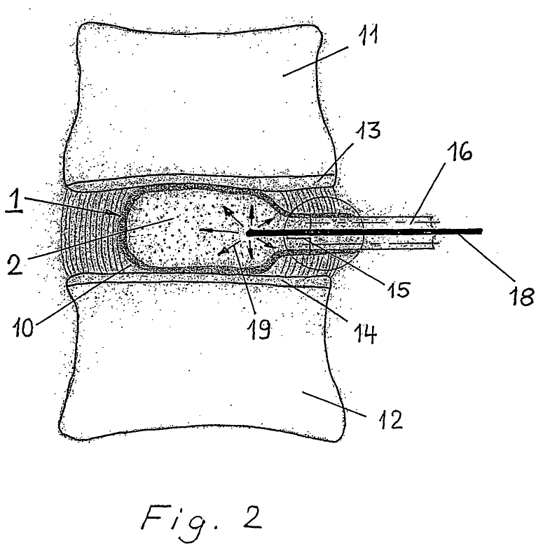

FIG. 2 is a longitudinal section of the intervertebral disk prosthesis of FIG. 1 when the flowable material is curing;

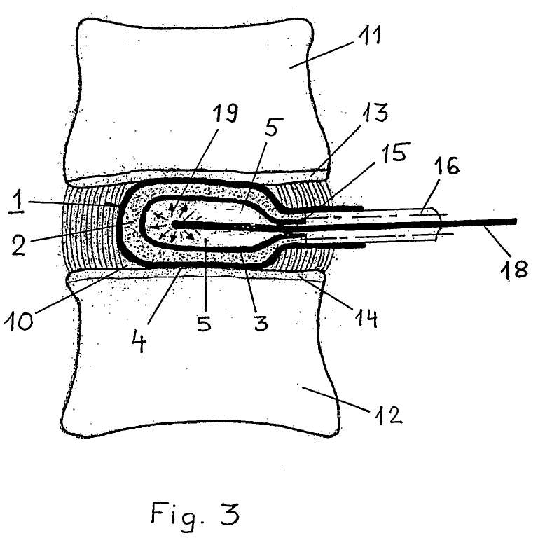

FIG. 3 is a longitudinal section of a double-wall intervertebral disk prosthesis;

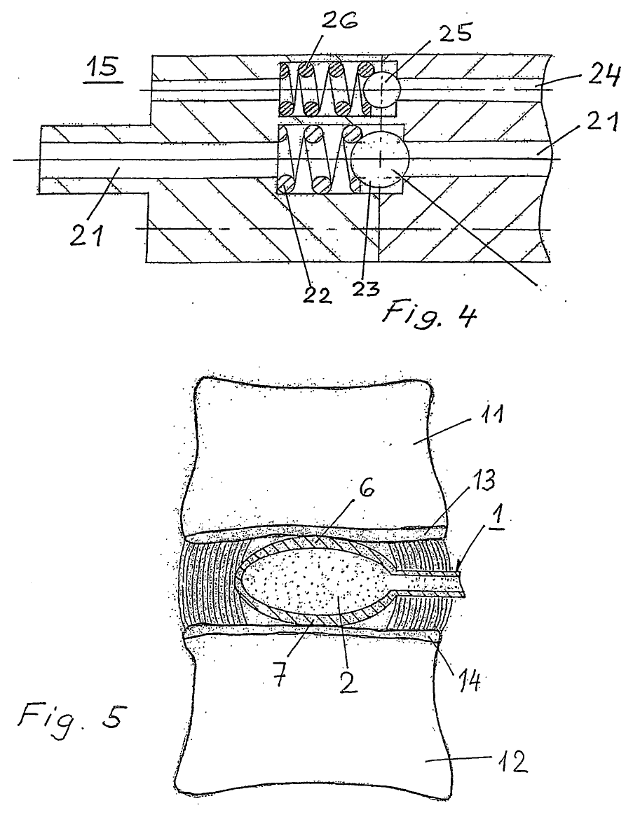

FIG. 4 is a longitudinal section of the filling valve of the intervertebral disk prosthesis; and

FIG. 5 is a longitudinal section of an intervertebral disk prosthesis comprising external surfaces of different thicknesses.

FIG. 1 shows the intervertebral disk prosthesis in the form of a nucleus replacement prosthesis in the state wherein the biocompatible pouch 1 already has been implanted in the intervertebral space 10 of two adjacent vertebras 11, 12 and wherein it is being filled through the valve 15 and the cannula 16 with a curable, flowable material 2 in the form of a hydrogel at the inside of the implanted biocompatible pouch 1 in the direction of the arrows 17. The filled pouch 1 remains centered in the intervertebral space 10 and rests against the two upper plates 13, 14 of the adjacent vertebras 11, 12.

FIG. 2 shows how the material 2 implanted in the biocompatible pouch 1 is cured by photo-polymerization by inserting a light guide 18 through the cannula 16 into said pouch. For that purpose the material 2 contains a radicals-generating photo-initiator. The light used for photo-initiation is indicated by the arrows 19 and is ultraviolet.

FIG. 3 shows a variation of the intervertebral disk prosthesis wherein the pouch 1 is double-walled and the material 2 is introduced between the two walls 3, 4, entailing a hollow center 5 of the intervertebral prosthesis.

To allow filling with material 2 both the single-wall as well as the double-wall variation of the intervertebral disk prosthesis, a special valve 15 shown in FIG. 4 is provided. Substantially this valve 15 comprises a central borehole 21 holding a ball 23 braced by a spring 22 and acting as a check valve, and a peripheral borehole 24 with a ball 25 braced by a spring 26 and also acting as a check valve. The central borehole 21 is used to fill the single-wall variant (shown in FIGS. 1 and 2), and the peripheral variant 24 is used to fill the double-wall variant (of FIG. 3). In the latter variant, the central borehole 21 may be used to introduce air or x-ray contrast means.

FIG. 5 shows a further variant of the intervertebral disk prosthesis wherein the pouch 1 comprises walls 6, 7 which shall rest against the upper plates 13, 14 of the adjacent vertebras 11, 12 and are made thicker than the wall zones elsewhere. At least the walls 6 and 7 of the pouch 1 consist of polycarbonate urethane (PCU) or of polycarbonate.

Several illustrative embodiments of the present invention are discussed below.

### EXAMPLE 1

45 g of polyethylene glycol diacrylate (PEGDA) having a molecular weight of 700 and 5 g of a copolymer of 2,6-dimethyl-3-vinylbenzoyl phosphine oxide (DMVBPO) and dimethyl acrylamide were dissolved in 50 g distilled water. This hydrogel was cured with blue light having a wavelength of 420 nm and an intensity of 2 watt/cm².

### EXAMPLE 2

40 g polyethylene glycol diacrylate (PEGDS) having a molecular weight of 700 and 5 g of a copolymer of 4-(VBPO) and dimethyl acrylamide were dissolved in 50 g distilled water. This hydrogel was cured with blue light having a wave length of 420 nm and an intensity of 2 watt/cm².

### EXAMPLE 3

45 g polyethylene glycol diacrylate (PEGDA) having a molecular weight of 750 and 5 g of a copolymer of 2,4,6-trimethylbenzoyl-phenyl-4-vinylphenyl phosphine oxide (TMBVPO) and dimethyl acrylamide were dissolved in 50 g distilled water, This hydrogel was cured with blue light having a wavelength of 420 nm and an intensity of 2 watt/cm².

1. An intervertebral disk prosthesis or nucleus replacement prosthesis comprising a bio-compatible pouch (1) receiving a curable, flowable material (2), said curable, flowable material including a material selected from the group consisting of monomers, comonomers, homopolymers, oligomers, and mixtures thereof,
wherein at walls (6, 7) designed to rest against upper plates (13, 14) of adjacent vertebras (11, 12), the pouch (1) is thicker than in remaining wall segments that are not designed to rest against the upper plates of adjacent vertebras.

2. The intervertebral disk prosthesis as claimed in claim 1, wherein the flowable material (2) contains a photo-initiator.

3. The intervertebral disk prosthesis as claimed in claim 2, wherein the photo-initiator absorbs light in the 340 to 420 nm range.

4. The intervertebral disk prosthesis as claimed in claim 2, wherein the photo-initiator is a selected from the group consisting of phosphine oxide and acylphosphine oxide.

5. The intervertebral disk prosthesis as claimed in claim 4, wherein the phosphine oxide is copolymerized with dimethylacryl amide.

6. The intervertebral disk prosthesis as claimed in claim 1, wherein the monomers, comonomers, homopolymers, oligomers or mixtures thereof are selected from the group of
(a) polyethylene glycols, including polyethyleneglycol (di)acrylates;
(b) N-vinylpyrrolidones; and
(c) vinyls, including vinyl alcohols; and
(d) styrenes.

7. The intervertebral disk prosthesis as claimed in claim 1, wherein a weight percent of water of the flowable material (2) is selected from the group of ranges consisting of 30 to 160% water and 40 to 90% water.

8. The intervertebral disk prosthesis as claimed in claim 1, wherein the flowable material (2) contains a hydrogel.

9. The intervertebral disk prosthesis as claimed in claim 1, wherein the pouch (1) has a double-walled structure and the flowable material (2) is introduced between two walls (3, 4) of the double-walled structure whereby a center (5) of the intervertebral disk prosthesis is hollow.

10. The intervertebral disk prosthesis as claimed in claim 1, wherein the pouch (1) is made of a memory-effect substance whereby said pouch shall assume a previously stored geometric shape at body temperature.

11. The intervertebral disk prosthesis as claimed in claim 1, wherein the flowable material (2) further includes a material selected from the group consisting of a polymerization catalyst and a polymerization accelerator.

12. The intervertebral disk prosthesis as claimed in claim 1, wherein the pouch (1) is made of a substance that is chemically identical to the material contained in the pouch.

13. The intervertebral disk prosthesis as claimed in claim 1, wherein, at its walls (6, 7) touching the upper plates (13, 14) of the adjacent vertebras (11, 12), the pouch (1) is made relatively thicker and preferably consists of a material selected from the group consisting of polycarbonate urethane (PCU) and polycarbonate.

14. A method for manufacturing an intervertebral disk prosthesis or a nucleus replacement prosthesis, comprising the steps of:
(a) implanting a bio-compatible pouch (1) in the intervertebral space (10) of two adjacent vertebras (11, 12),
(b) introducing a curable, flowable material (2) inside the implanted, biocompatible pouch (1), said flowable material including materials selected from the group consisting of monomers, comonomers, oligomers and mixtures thereof, the pouch (1) when filled remaining centered in the intervertebral space (10); and,
(c) curing in situ the curable, flowable material (2) that is disposed within the pouch (1).

15. The method as claimed in claim 14, wherein the material (2) is cured by photo-polymerization using visible or ultraviolet light.

16. The method as claimed in claim 14, wherein the pouch (1) is inflated with air between the steps (a) and (b).

17. The method as claimed in claim 14, wherein, between the steps (a) and (b), the pouch (1) is filled with an x-ray contrast means.

18. The method as claimed in claim 14, wherein the flowable material (2) is cured by auto-polymerization.

19. The method as claimed in claim 14, wherein the material (2) is introduced substantially at no excess pressure into the pouch (1).

20. The method as claimed in claim 14, wherein the material (2) is introduced into the pouch (1) at an excess pressure, said excess pressure being selected from the group of ranges consisting of less than 3 atm and less than 1.1 atm.

21. (canceled)
